(19) United States
(12) Patent Application Publication
(10) Pub. No.: US 2012/0180167 A1
(43) Pub. Date: Jul. 12, 2012

(54) MEDIUM FOR CULTIVATING TRUFFLES, TRUFFLE MYCELIUM, AND TRUFFLE FRUITING BODY

(76) Inventor: Ken USAMI, Niigata (JP)

(21) Appl. No.: 13/032,938

(22) Filed: Feb. 23, 2011

(30) Foreign Application Priority Data
Jan. 7, 2011 (JP) 2011-002460

(51) Int. Cl. A01H 15/00; A01G 1/04

(52) U.S. Cl. 800/297; 71/5

(57) ABSTRACT

The purpose is to provide the truffle mycelium and fruiting body cultivated by the medium that are developed for cultivating the truffles with a very high radical oxygen scavenging capacity. A medium for cultivating truffles includes a mixture of at least one carbohydrate source selected from a group consisting of rice bran, wheat bran, and glucose, and at least one edible ingredient selected from a group consisting of burdock, nuts, and berries. A weight ratio of the edible ingredient to the carbohydrate source is between 0.5 and 5. As for one of the ingredients, it is preferable that the burdock is granulated. The medium may exist in a liquid state. By injecting truffle seeds for cultivation into such a medium, the truffle mycelium and fruiting body with the high reactive oxygen scavenging capacity could be provided.

### TECHNICAL FIELD

This invention relates to a medium for, cultivating truffles, and more precisely, a medium, which allows to produce the truffle mycelium and truffle fruiting body with the high reactive oxygen scavenging capacity.

### BACKGROUND ART

The reactive oxygen has been considered as one of the contributory factors for aging and diseases. In recent years, it is believed to be desirable to restrain or eliminate it. The known components to remove the reactive oxygen are, for example, β-glucan polysaccharide, vitamin C, Vitamin E, Saponin, Catechin, Flavonoid, Tannin, β-carotene, Polyphenol, Anthocyanin, etc.

Apropos, mushrooms are known that they have various bioactive properties and it is also known that the mushroom has an effect to moderate damages caused to a living body by life circumstance and natural environment.

For example, Kabanoanatake contains enzyme such as Superoxide Dismutase (SOD), which is known to be effective in the treatment and prevention of dementia, hepatitis, diabetes, cerebral vascular disorder, cardiac infarction and cancer, potentially caused by the a slight amount of the reactive oxygen generated in the process of energetic metabolism of the living body.

Also, truffles are known to be effective on certain conditions such as the improvement of immunity and hyperlipidemia. The well-known truffles are Perigord truffles from France (also known as black truffle, scientific name: Tuber melanosporum), and the Italian white truffles (scientific name: Tuber magnatum pico). In Japan, Kuroamimeseiyoushouro (scientific name: Tuber aestivum) is also widely known.

In addition, the Patent Document 1 discloses the artificial cultivation method of white truffle. The Patent Document 2 discloses that the extracted substance (truffle extract) of truffle mycelium and fruiting body, using a solvent such as ethanol, is effective in reducing the aging of the organism. Patent Document 3 discloses that the truffle extract is effective in the prevention of the immunological deterioration as well as moisture retention of the skin. Patent Document 4 discloses the truffle extract is effective to the secretagogue action of the secretory form immunoglobulin A (IgA), improvement of the immunity balance and the anti-hyperlipidemia.

As illustrated in the aforementioned patent documents 2, 3 and 4, truffes can be, not only consumed as delicacies, but also can be potentially provided as health promoting food, which is utilized for prevention of various diseases as well as the protection and improvement of bodily functions.

However, the methods disclosed in Patent Documents 2 and 3 are just simply using the fruiting body of truffles as one of the basic ingredients, which are conventionally manufactured and commercially available at the today's market, and these are not intended to improve or further study of these traditional truffle mycelium and medium (also known as “culture medium”) which produces the truffle fruiting body.

Note that the typical truffle medium is prepared with a mixture of wood sawdust (wood debris or wood chips), rice bran and other nutrients. Then a hole is made in the medium and a spawn (seed) of the fungus is injected in the culture medium and the medium is kept in the low temperature for cultivation. After about one week, the white fluffy fungal filament starts to grow in the entire medium. In one to two months, the fungal filaments are entirely infested. (i.e. the truffle mycelium is formed.) In three to four months, 2 to 3 cm of the truffle fruiting bodies are formed on the surface of the medium covered by the white aerial hypha.

Since the truffle fruiting bodies, cultivated as described above and collected from the culture medium, do not include an inedible element or the sawdust, it can be offered to the market as food delicacy. However, if we try to extract the truffle mycelium from the medium in the middle of the cultivation process, isolating the truffle mycelium completely from the inedible element as sawdust is not possible; hence, using it as a basic ingredient of the health promoting food requires the subsequent post-processing.

Although truffles and truffle extracts by the conventional method may have reactive oxygen scavenging capacity (antioxidative activity), improvements are still required. Furthermore, as for the commonly used medium (culture medium), there is no evidence that the medium itself has the remarkable antioxidative capacity.
Patent Document 1; JP Patent Publication (unexamined) No. 10-127164
Patent Document 2; JP Patent Publication (unexamined) No. 2002-249438
Patent Document 3; JP Patent Publication (unexamined) No. 2006-69969
Patent Document 4; JP Patent Publication (unexamined) No. 2010-222265
Non-Patent Document 1: C. Folch-Cano and three other authors, “Antioxidant activity of inclusion complexes of tea catechins with β-cyclodextrins by ORAC assays”, Food Research International, 43 (2010), 2039-2044

### SUMMARY OF THE INVENTION

The present invention is developed in a view of the aforementioned circumstances, and its purpose is to provide the truffles (truffle fruiting body) that have the high antioxidative capacity.

Another purpose of the present invention is to provide a medium for producing truffles and truffle mycelium having high antioxidative capacity.

After a diligent study, the inventor discovered that, when an edible ingredient having high Oxygen Radical Absorbance Capacity (ORAC), or high antioxidative potency, is used as an ingredient of the medium replacing the sawdust used solely in the conventional method, not only the medium but also the injected spawn (seeds) of truffles absorbing said ingredient as well as the truffle mycelium and truffle fruiting body cultured on said medium drastically increase their reactive oxygen scavenging capacity comparing to the existing truffles.

More specifically, this invention consists of, for example, the following characteristics and components. A medium for cultivating truffles, said medium comprises a mixture of at least one carbohydrate source selected from a group consisting of rice bran, wheat bran, and glucose, and at least one edible ingredient selected from a group consisting of burdock, nuts, and berries, wherein a weight ratio of said edible ingredient to said carbohydrate source is between 0.5 and 5.

Here, as the aforementioned edible ingredients, at least one of burdock, nuts (e.g., Walnuts), and berry (e.g., blueberry) has been selected. It is well known that the ORAC values are very high in all of these fruits and vegetables.

Additionally, with the present invention, one of the traditional ingredient sawdust is not at all employed and the edible ingredients have become an alternative to the sawdust. This enables the cultivated truffle mycelium is also edible. Therefore, the range of utilization of the truffle mycelium can be extended significantly.

Considering the costs of the basic ingredients, it is preferable that they contain burdock. As for increasing the effect of the invention mentioned in the followings, it is desirable to use the granulated burdock in 1 mm to 10 mm in a grain diameter, for example. The grain size of less than 1 mm becomes very close to its powdered state, and the medium using such a small size of burdock has poor ventilation, therefore it is not suitable for cultivating the mycelium. On the other hand, if the grain size goes more than 10 mm in diameter, it does not mix well with the carbohydrate source and it fails to develop an adequate medium.

Moreover, in mixing the carbohydrate source such as rice bran and the edible ingredient, it is preferable to maintain the edible ingredient to be mixed with the weight ratio of 0.5 to 5 to the carbohydrate source (that is, the weight of the carbohydrate source as 1). Also, even more preferably, the edible ingredient is to be mixed with the weight ratio of 1 to 2. If the weight ratio of the edible ingredient is less than 0.5, it is undesirable as it causes nutritional disorder in the process of truffle cultivation. On the contrary, if the weight ratio of the edible ingredients would be more than 5, it is also undesirable as it prolongs the incubation time of truffles to the unacceptable level.

The kinds of truffles, which may be applied to the present invention, are not specially limited to any specific species. But the seeds of the Black Truffle (scientific name: Tuber melanosporum), the seeds of the White Truffle (scientific name: Tuber magnatum pico) and the seeds of Kuroamimeseiyoushouro (scientific name: Tuber aestivum) may be listed, for example.

According to the present invention, it is possible and quite easy to cultivate the truffle mycelium and the truffle fruiting body with very high reactive oxygen scavenging capacity.

### BRIEF DESCRIPTION OF THE DRAWING

FIG. 1 is a graph demonstrating the decreased fluorescence intensity of truffle culture cultivated by the present invention, monitored by the analysis of ORAC method.

### DETAILED DESCRIPTION OF PREFERRED EMBODIMENTS

At first, we will explain about the medium of the present invention and the culture cultivated by injecting the truffle seeds into such a medium.

### First Embodiment

As a condition of the first embodiment of the present invention, we produced the medium by mixing 0.5 kg of rice bran and 0.5 kg of burdock, then injected 10 g of truffle seed and mixed them more, and transferred this medium in a sterile container and stored the container in a clean bench under the sterile environment at a temperature of 20 Celsius for 60 days. After the 60 days of incubation, we collected 1 g of the culture in a test tube with 20 ml volume and added 10 ml of 80 percent methanol and extracted them by heating on a hot plat 100 Celsius to 105 Celsius for 30 minutes. The extracted liquid was removed from the test tube and maintained in the centrifugal separation process for 10 minutes under 8000 rpm to collect the supernatant liquid. The collected supernatant was stored frozen at minus 20 Celsius.

Furthermore, for the evaluation of the ORAC method as mentioned below, we have adjusted the concentration of the aforementioned supernatants, enabling the proper evaluation of this analytical method. More specifically, we collected the 10 μl of the refrigerated supernatant and heated to 25 Celsius and diluted it by adding 999 μl of the 80 percent methanol solution.

### Second Embodiment

As a condition of the second embodiment of the present invention, the only condition, which was different from that of the first embodiment, is the weight ratio of the ingredients to prepare the medium. The quantities were changed to 0.5 kg of rice bran and 1.0 kg of burdock. Since the second embodiment is conducted as the first embodiment except the weight ratio, the explanation about the other same conditions is omitted.

### Comparative Examples

In order to verify the function effect of the truffles culture of the present invention, truffle culture was carried out using conventional method as a comparative example. Specifically, the medium of the comparative example was obtained by mixing 0.5 kg of rice bran with 1.0 kg of sawdust. Except the ingredients, the comparative example was prepared in the same way as the first practical example, thus, the explanation is omitted.

### (Affirmation of the Advantageous Effects on Oxygen Radical Absorbance Capacity ORAC)

ORAC (Oxygen Radical Absorbance Capacity) is the index which demonstrates the antioxidative potency, and was developed by the researchers of the U.S. Department of Agriculture (USDA) and the National Institute on Aging in 1992. This ORAC assay is excellent especially for the analysis of antioxidative capacity in foods. The ORAC database based on such an analysis offers a wide range of collection from natural ingredients such as vegetables and fruits to the processed foods.

### (The Principle of ORAC Analysis)

The measurement of the fluorescence intensity of the fluorescent substances disassembled by a certain amount of generated active oxygen species makes a diminishing curve over time. When the antioxidative substance coexists to this reaction system, it reduces the declining speed of fluorescence intensity.

Based on this measurement principle, the difference (called net AUC) between the Area Under the Curve (AUC) of the fluorescence intensity in the presence of the specimen material (or the reference material) and the AUC without the presence of the specimen material (the blank condition) was calculated. Concerning the net AUC of the specimen material, it is calculated the relative value to the net AUC of the known density of the reference material (Trolox: registered trade mark). The Oxygen Radical Absorbance Capacity of the specimen material is established by the Trolox density converted based on the relative value. The unit of net AUC is μmol TE/g.

The aforementioned evaluation was conducted for the first and second embodiments and the comparative example. For this evaluation, 1 mM of Trolox solution is prepared as the reference material and Trolox solutions, whose concentration gradually are modified by 80 percent methanol from the range of 0.782 μM to 50 μM, are also prepared.

The green dye, flourescein is used as the fluorescent pigment for the aforementioned measurement and 2, 2′-azo-bis (2-amidinopropane) dihydrochloride AAPH is used as the reactive oxygen scavenging reaction indicator. To monitor the fluorescence intensity, a black plate with 96 halls for holding test tubes (trade name: OptiPlate-96, PerkinElmer Inc.), and Multi Label Reader (trade name ARVOX, PerkinElmer Inc.) are used as the analytical equipments. Furthermore, we referred to the aforementioned non-patent Document 1 for the procedures of this evaluation.

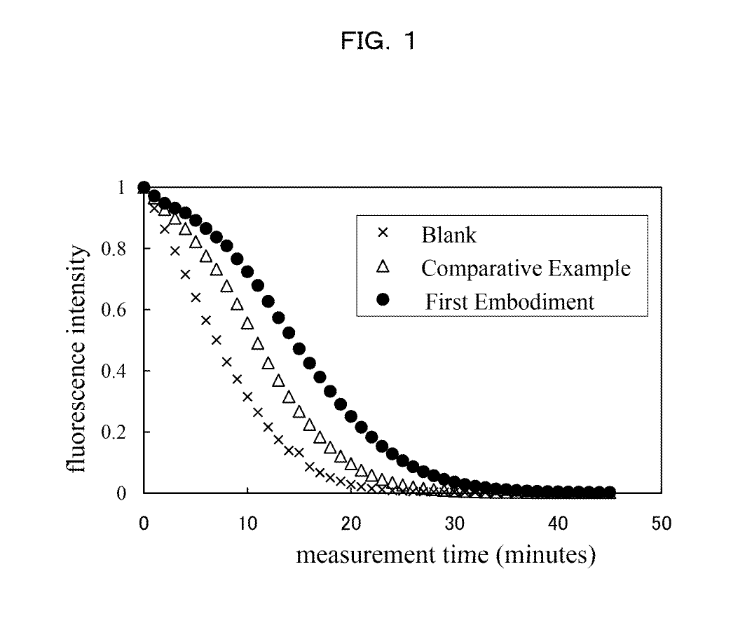

In FIG. 1, it is demonstrated that the monitoring results of the decline of fluorescence intensity of the blank condition, the comparative example as well as the first embodiment of the present invention. Additionally, in Table 1 and Table 2, the analytical results (AUC and net AUC) of the blank condition, the reference material in each concentration (Trolox solution), the comparative example and the first embodiment of the present invention. As mentioned, since the truffle culture of the comparative example and the first embodiment is 1000 times more diluted by ethanol for this evaluation, we estimate that the net AUC (Antioxidative potency) of actual truffle culture will be 1000 times more than the result of this evaluation.

TABLE 1

| | | Trolox | Trolox | Trolox | Trolox | Trolox | Trolox | Trolox |
|---|---|---|---|---|---|---|---|---|
| | Blank | No. 1 | No. 2 | No. 3 | No. 4 | No. 5 | No. 6 | No. 7 |
| Trolox Concentration | 0 | 0.782 | 1.563 | 3.125 | 6.25 | 12.5 | 25 | 50 |
| AUC | 7.542 | 9.228 | 10.6 | 12.37 | 15.03 | 18.74 | 23.59 | 28 |
| net AUC | 0 | 1.686 | 3.056 | 4.827 | 7.488 | 11.19 | 16.05 | 20.46 |

TABLE 2

| | Comparative Example | First Embodiment |
|---|---|---|
| AUC | 9.844 | 14.05 |
| net AUC | 2.3 | 6.5 |

Based on the results comparing to the truffle culture incubated in the conventional method as in the comparative example, the culture prepared by the present invention demonstrates the significant high antioxidative potency.

More specifically, the culture incubated the medium of the first embodiment with the weight ratio of 1:1 of rice bran and burdock respectively, demonstrated approximately three times greater antioxidative potency (net AUC) than that of the comparative example.

Furthermore, although it is not illustrated in the FIGURE, in the case of the culture cultivated in the medium with the weight ratio of 2:1 of rice bran and burdock respectively, as stated in the second embodiment, it also demonstrated a higher antioxidative potency than that of the comparative example.

In the present invention, the medium for cultivating truffles generally exists in a solid state, but it may exist in a liquid state. In that case, both the medium and the truffle culture are in a liquid state. For example, the liquid medium is poured into a tank. Then, the seed is added into the medium resulting in a liquid truffle culture in the tank. Compared to the solid medium, which is not poured into a tank and usually is exposed to outside environment (not clean and not pure), making the medium in a liquid state and keeping it in a tank results in a purer truffle culture without contamination. Thus, the medium in liquid state allows cultivating a purer liquid truffle culture in a tank. Said truffle culture may be used as a basic ingredient for processing the health promoting foods. For example, the medium used in liquid state may comprise a mixture of a liquid burdock extract made by an ethanol extraction or a thermal extraction, and Rice bran, with the preferred weight ratio, and may be poured in a tank. Then, a seed of the fungus may be injected into the liquid medium in the tank.

As noted above, truffles, will not only be considered as a food delicacy, it will also be utilized as a useful functional food ingredients. The truffle mycelium and fruiting body of the present invention are, comparing to the ones available at the today's market, it can be considered as one of the ingredients for functional foods or more value added ingredient since they possess already a high antioxidative potency. Consequently, the present invention has a very high value of industrial application.

What is claimed is:

1. A medium for cultivating truffles, said medium comprising a mixture of at least one carbohydrate source selected from a group consisting of rice bran, wheat bran, and glucose, and at least one edible ingredient selected from a group consisting of burdock, nuts, and berries, wherein said edible ingredient has a weight ratio relative to the carbohydrate source between 0.5 and 5.

2. The medium for cultivating truffles according to claim 1, wherein said edible ingredient has the weight ratio relative to the carbohydrate source between 1 and 2.

3. The medium for cultivating truffles according to claim 1, wherein said edible ingredient is a granulated burdock.

4. The medium for cultivating truffles according to claim 3, wherein said granulated burdock has a grain diameter between 1 mm and 10 mm.

5. The medium for cultivating truffles according to claim 1, wherein said medium is in a liquid state.

6. A truffle mycelium being produced by injecting a truffle seed to the medium according to claim 1.

7. A truffle fruit body being produced by injecting a truffle seed to the medium according to claim 1.
